(19) United States
(12) Patent Application Publication
(10) Pub. No.: US 2021/0261643 A1
(43) Pub. Date: Aug. 26, 2021

(54) INHIBITORS OF P-TEFB MEDIATED TRANSCRIPTION

(71) Applicant: The Regents of the University of California, Oakland, CA (US)

(72) Inventors: Koh Fujinaga, San Francisco, CA (US); Boris Peterlin, San Francisco, CA (US); Marie Leoz, Rouen (FR)

(73) Assignee: The Regents of the University of California, Oakland, CA (US)

(21) Appl. No.: 17/220,152

(22) Filed: Apr. 1, 2021

Related U.S. Application Data
(63) Continuation of application No. PCT/US2019/054134, filed on Oct. 1, 2019.
(60) Provisional application No. 62/739,425, filed on Oct. 1, 2018.

(51) Int. Cl. C07K 14/725; A61P 31/18

(52) U.S. Cl. CPC C07K 14/7051; A61P 31/18; C07K 2319/03; C07K 2319/02; C07K 2319/035; A61K 38/00; C07K 2319/20; C07K 2319/32; C07K 2319/715; C07K 2319/90; C07K 2319/09

(57) ABSTRACT

Novel chimeric proteins may be used to inhibit transcriptional activities that are mediated by transcription factor interactions with P-TEFb. The chimeras contain elements that recruit the target transcription factor, maintain CDK9 in an inactive state, and competitively inhibit P-TEFb binding to the transcription factor. The chimeras may be configured for inhibition of HIV Tat mediated transcription and thus provide a novel means of preventing reactivation of integrated HIV, providing a new tool for emerging “block and lock” HIV cure strategies.

### CROSS-REFERENCE TO RELATED APPLICATIONS

This application is a 35 USC § 371 national stage application of International Patent Application Number PCT/US2019/054134, entitled “Inhibitors of P-TEFb Mediated Transcription,” filed Oct. 1, 2019, which claims the benefit of priority to U.S. Provisional Patent Application Ser. No. 62/739,425 entitled “Transcription Factor-Specific P-TEFb Inhibitors,” filed Oct. 1, 2018, the contents of which applications are hereby incorporated by reference.

### STATEMENT REGARDING FEDERALLY SPONSORED RESEARCH OR DEVELOPMENT

This invention was made with government support under grant number R01 AI049104 awarded by the National Institutes of Health. The government has certain rights in the invention.

The instant application contains a Sequence Listing which has been submitted electronically in ASCII format and is hereby incorporated by reference in its entirety. Said ASCII copy, created on Oct. 1, 2019, is named UCSF069PCT_SL.txt and is 7,693 bytes in size.

### BACKGROUND OF THE INVENTION

The P-TEFb transcriptional activator is a highly regulated activator of transcription elongation and gene expression. The P-TEFb is comprised of two subunits: a cyclin T1 (comprising either CycT1 or CycT2) and cyclin-dependent kinase 9 (CDK9). P-TEFb is implicated in various conditions wherein gene expression contributes to or drives the pathological condition. For example, P-TEFb is implicated in HIV infection, cancer, and conditions such as cardiac hypertrophy.

In the case of HIV, treatment with combination antiretroviral therapy (cART) leads to efficient suppression of HIV replication, but HIV persistence in latently infected cells remains an obstacle to cure. Even under cART treatment, residual HIV replication can arise and ultimately lead to the emergence of resistance mutations and viral escape. Targeting diverse steps of the viral cycle is the most efficient way to prevent viral escape. Currently, viral entry, reverse transcription, integration and maturation steps have been targeted by cART.

Blocking HIV transcription could potentially be utilized to prevent sporadic reactivation of integrated HIV that may contribute to HIV persistence, reservoir replenishment and chronic inflammation. Suppressing residual HIV transcription is also the goal of the emerging “block and lock” HIV cure strategies, which aim at deepening HIV latency so that integrated proviruses remains definitively locked in the infected cells. Previous studies have demonstrated that transcriptional inhibition can potentially be targeted. For example, the use of TAR decoys to inhibit HIV replication has been demonstrated, for example, as described in Michienzi et al., A nucleolar TAR decoy inhibitor of HIV-1 replication. PNAS. 2002; 99(22):14047-52. Additionally, the use of dominant-negative Tat mutants to suppress HIV transcription has been explored, for example, as described in Green et al., Mutational analysis of HIV-1 Tat minimal domain peptides: identification of trans-dominant mutants that suppress HIV-LTR-driven gene expression. Cell. 1989; 58(1):215-23. However, to date, no efficient transcription inhibitor to prevent expression of the integrated provirus has been demonstrated or is clinically available. Accordingly, there is a need in the art for novel and potent inhibitors of HIV transcriptional activation.

Similarly, there is a need in the art for transcriptional inhibitors to treat other conditions where P-TEFb is implicated, such as cancer, cardiac hypertrophy, inflammation, and infectious diseases other than HIV.

### SUMMARY OF THE INVENTION

Demonstrated herein is a novel and effective strategy for inhibiting transcription factor-P-TEFb complexes that activate transcription. In a first aspect, the scope of the invention encompasses novel therapeutic proteins for the treatment of conditions mediated by transcriptional activities of P-TEFb. The novel therapeutic compositions of the invention may be applied in the inhibition of transcriptional activities mediated by P-TEFb and a selected target transcription factor wherein, wherein the target transcription factor is any transcription factor that forms a complex between itself and P-TEFb and such complex is implicated in the activation of transcription. In various embodiments, the transcription factor targets may include HIV Tat, NFkB, cMyc, MyoD, STAT-family transcription factors, Class II transactivator (CIITA), Autoimmune Regulator (AIRE), Eleven Nineteen Leukemia (ENL), AF4 proteins implicated in acute leukemia, estrogen receptors, androgen receptors, MEF2, and HTLV1 Tax.

The therapeutic compositions of the invention encompass a multifunctional agent, wherein the agent achieves the following three functions:
recruiting the target transcription factor to the trifunctional molecule;
maintaining the CDK9 subunit of P-TEFb in an inactive state; and
inhibiting binding of the target transcription factor to P-TEFb to activate P-TEFb.

By the administration of the trifunctional agent, aberrant or pathological P-TEFb mediated activation of transcription may be inhibited, for the treatment and prevention of any number of conditions.

In one aspect, the scope of the invention encompasses chimeric fusion proteins for use in inhibiting a transcriptional process wherein the process is mediated by P-TEFb and a target transcription factor, the target transcription factor being any transcription factor that mediates transcription by interactions with P-TEFb. In one aspect, the scope of the invention encompasses chimeric fusion proteins for use in a method of treating HIV infection.

### BRIEF DESCRIPTION OF THE DRAWINGS

FIGS. 1A, 1B, and 1C. Fusion Protein Configurations. FIG. 1A: Fusion protein design for HIV transcriptional inhibition. The fusion protein comprises a first element which recruits HIV Tat to the fusion proteins, such as an arginine rich motif from TAT or HEXIM1. The second element comprises a CDK9 inhibitory domain, such as the inhibitory domain of HEXIM1. The third element comprises a TAT domain that binds to P-TEFb. Optional linker sequences (L1 and L2) are present between the elements, for example, comprising SEQ ID NO: 5 repeats. FIG. 1B: Fusion protein design for inhibiting transcription in other P-TEFb mediated systems, wherein the first element is an element that recruits the target transcription factor. FIG. 1C: In an alternative implementation, the first element and first linker (L1) is omitted, while the inhibitory domain and P-TEFb-binding domains are present and sufficient to inhibit transcription.

FIGS. 2A, 2B, and 2C. A HEXIM1-Tat fusion peptide inhibits gene expression from the HIV promoter. FIG. 2A. Structure of the HEXIM1-Tat fusion peptides used in the study. The functional domains used from HEXIM1 and Tat include a HEXIM1 Arginine Rich Motif (ARM, black box, residues 150-162) that binds RNA, a HEXIM1 inhibitory domain (ID, light grey box, residues 200-211) that inhibits CDK9 through a PYNT motif, and Tat transactivation domain (AD, dark grey box, residues 1-48) that binds to P-TEFb. The conceptual schemes are not drawn to scale. FIG. 2B. Transient expression of HT1 inhibits Tat-induced LTR-driven Luc expression. Upper panel: a schema depicts the reporter assay. A luciferase reporter gene (Luc) is under the control of the HIV promoter (P-HIV) and can be activated by ectopically expressed Tat. The Luc assay was used to titrate the inhibitory effect of HT1-3 on P-HIV transactivation by Tat. Middle panel: increasing amounts of m:HT1, m:HT2 or m:HT3 expressing plasmid (pHT) were co-transfected in 293T cells with a plasmid (pTat) expressing f:Tat and another (pLTR-Luc) expressing Luc under the control of the HIV promoter. Luc activity was plotted as % activity relative to control (empty vector used instead of pHT), depending on the transfected pHT:pTat ratio. Error bars in the graph represent standard deviation from triplicate experiments. FIG. 2C. Transient expression of HT1 inhibits Luc expression from HIV-1 NL43ΔenvLuc. Top schema depicts the reporter assay. A luciferase reporter gene (Luc) is inserted in a replication-defective HIV-1 molecular clone (pNL43ΔenvLuc) in which Luc and Tat are under the control of the HIV promoter (P-HIV). A Luc assay was used to titrate the inhibitory effect of HT1-3 on P-HIV transactivation by Tat. Bottom: increasing amounts of pHT1 were co-transfected in 293T cells with pNL43ΔenvLuc. Luc activity was plotted as % activity relative to control (empty vector used instead of pHT), depending on the transfected amounts of pHT1. Error bars in the graph represent standard deviation from triplicate experiments.

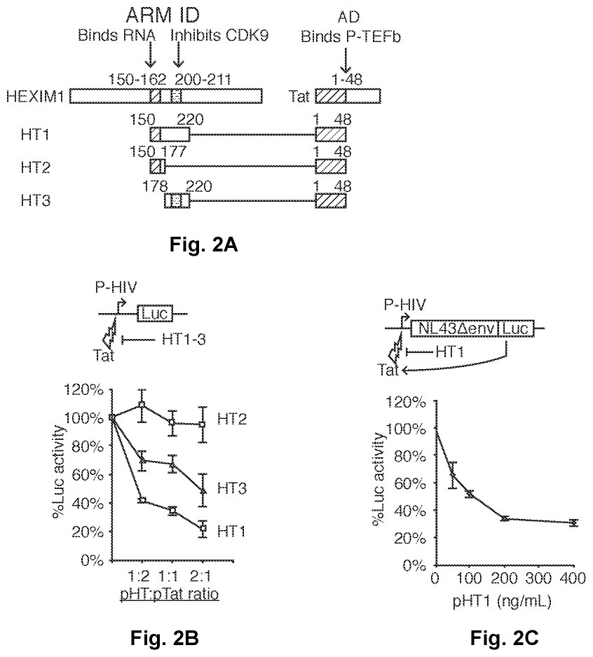

FIGS. 3A, 3B, 3C and 3D. HT1 prevents Tat from bringing active P-TEFb to TAR. FIG. 3A: HT1 inhibits the kinase activity of P-TEFb subunit CDK9. m:HT1 and/or f:Tat were transiently expressed in 293T cells. Cell lysates were used for IP using anti-Myc Ab (lanes 2-4), or control IgG (lane 1). Immunoprecipitates were incubated with ATP and recombinant GST-CTD proteins for in vitro kinase assay. Six replicate experiments were performed, and mean relative kinase activities are shown in the bar graph to the right. FIG. 3B: HT1 binds to TAR. m:HT1 or m:Tat (or empty vector, EV, as a control) was transiently co-expressed with TAR RNA-expressing pU16TAR in 293T cells. Cell lysates were used for IP using anti-Myc Ab or control IgG. RNA was purified from the immunoprecipitates and submitted to RT-qPCR using TAR-specific primers. Relative TAR enrichment was calculated as qPCR count using anti-Myc Ab minus using IgG, and normalized to EV. Error bars represent standard deviation from triplicate qPCR assays. FIG. 3C: The amounts and standard deviations of immunoprecipitated TAR RNA were normalized to the respective amount of m:HT1 or m:Tat detected in the input lysate. Error bars represent standard deviation from triplicate qPCR assays. FIG. 3D: HT1 competes with Tat for TAR binding. m:HT1 and/or f:Tat were transiently co-expressed with TAR RNA-expressing pU16TAR in 293T cells. Cell lysates were used for IP using anti-Myc Ab or control IgG. RNA was purified from the immunoprecipitates and submitted to RT-qPCR using TAR-specific primers (upper panel). Relative TAR enrichment was calculated as qPCR count using anti-Myc Ab minus using IgG, and normalized to EV. Error bars represent standard deviation from triplicate qPCR assays.

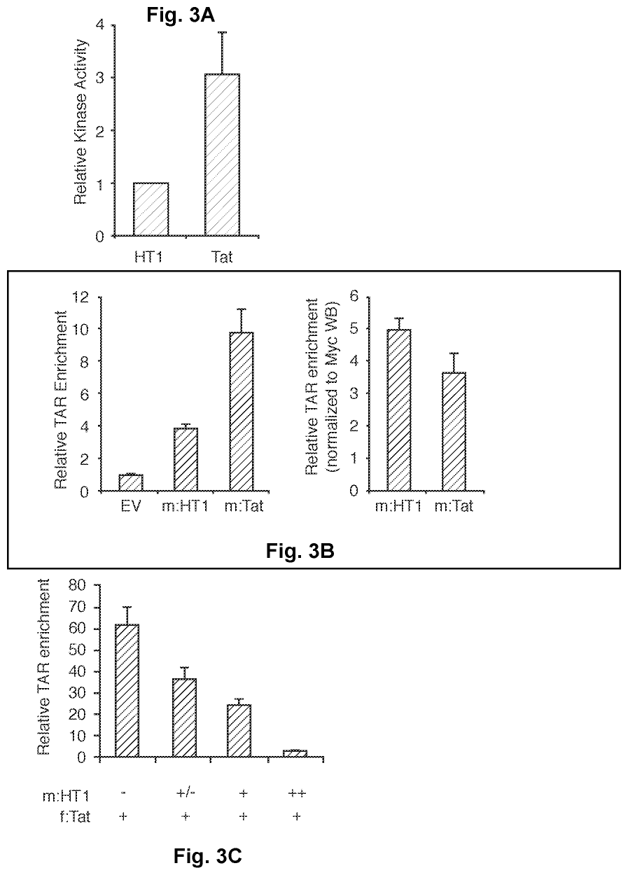

FIGS. 4A and 4B. Stable expression of HT1 inhibits HIV reactivation. FIG. 4A: Stable expression of HT1 inhibits HIV reactivation from 2D10 cells. 3f:HT1 was stably expressed in 2D10 cells (D-HT cells). FACS analysis of GFP positive cells showed that HIV reactivation by PMA, SAHA or JQ1 was significantly impaired in D-HT cells when compared to 2D10 cells, as determined by a student t-test. *** represent differences with p<E−03 (p=4.75E−05, p=4.35E−05 and p=7.62E−05 respectively). Error bars represent standard deviation from triplicate FACS analysis. FIG. 4B: Stable expression of HT1 inhibits HIV reactivation from J-Lat 9.2 cells. 3f:HT1 was stably expressed in J-Lat 9.2 cells (L-HT cells). FACS analysis of GFP positive cells showed that HIV reactivation by PMA was significantly impaired in L-HT cells when compared to J-Lat 9.2 cells, as determined by a student t-test. *** represent differences with p<E−03 (p=5.62E−05). Error bars represent standard deviation from triplicate FACS analysis.

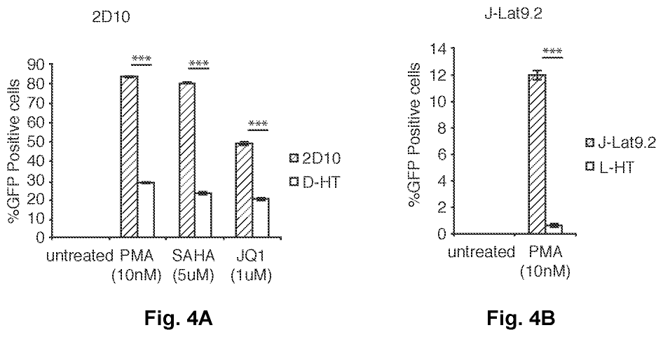

FIGS. 5A, 5B, 5C, and 5D. Stable expression of HT1 inhibits HIV replication. FIG. 5A. Stable expression of HT1 inhibits HIV-1 replication in C-HT cells. 1E+06 HT1-expressing CEM-HT cells and e control CEM cells were challenged by HIV-1 NL43 infection. FIG. 5B. Stable expression of HT1 inhibits HIV-1 replication in MO-HT cells. 1E+06 HT1-expressing MOLT4-HT cells, and control MOLT4 cells were challenged by HIV-1 NL43 infection. FIG. 5C. Stable expression of HT1 inhibits HIV-1 replication in MT-HT cells. 1E+06 HT1-expressing MT4-HT cells, and control MT4 cells were challenged by HIV-1 NL43 infection. In each of FIGS. 5A, 5B, and 5C, virus production was assessed by Gag p24 ELISA in supernatants when cells were passaged at days 0, 2 and 4, and plotted. Error bars represent standard deviation from triplicate experiments. FIG. 5D: Stable expression of HT1 doesn't inhibit early steps of HIV-1 infection in C-HT, MO-HT, and MT-HT cells. Single-round infection assays were performed in the same conditions as in FIG. 5A-C above, but using replication-defective HXB2 Env-pseudotyped HIV-1 NL43ΔenvLuc. The relative amount of HIV DNA integrated in the cells' genomic DNA was assessed after 24 h by qPCR. Error bars represent standard deviation from triplicate experiments.

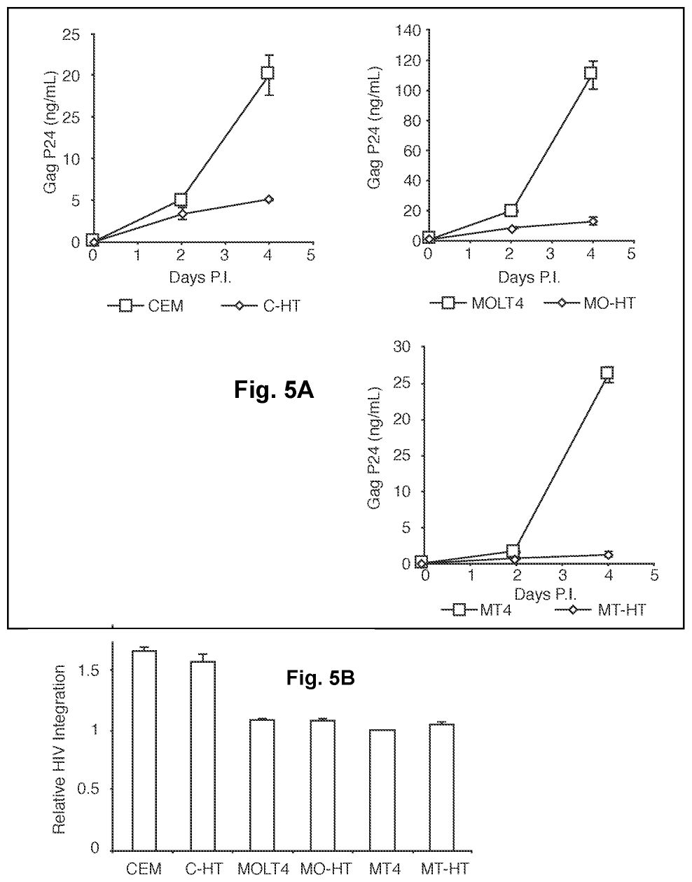

FIGS. 6A and 6B. FIG. 6A: Activity of various chimeras on Tat-induced LTR-driven Luc expression. As in FIG. 2B, pLTR-Luc was co-transfected with pTat and a pHT plasmid for expression of the indicated chimera in 293T cells (pHT:pTat ratio=1:2). Luc activity was plotted as % activity relative to control (EV=empty vector used instead of pHT). Error bars in the graph represent standard deviation from triplicate experiments. FIG. 6B: Transient expression of HT1 inhibits Tat-induced LTR-driven Luc expression in NIH1 cells, which stably carry an LTR-Luc reporter gene. pTat and a pHT plasmid for expression of the indicated chimera were co-transfected in NH1 cells (pHT:pTat ratio=1:2). Luc activity was plotted as % activity relative to control (EV=empty vector used instead of pHT). Error bars in the graph represent standard deviation from triplicate experiments.

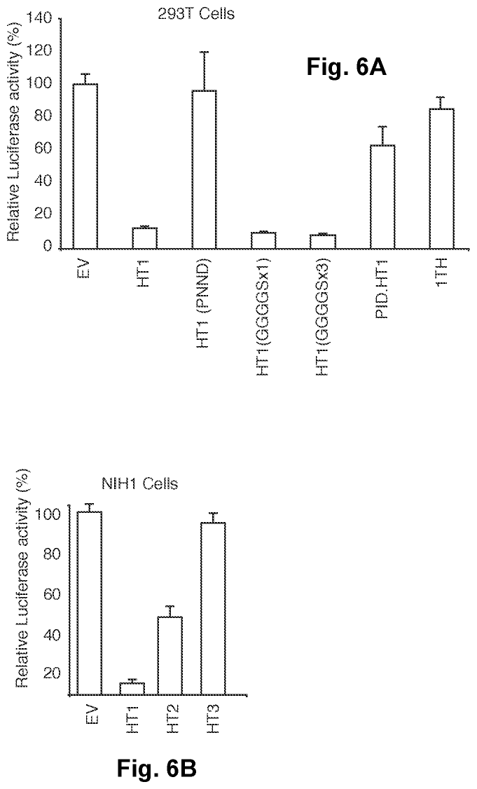

### DETAILED DESCRIPTION OF THE INVENTION

In a first aspect, the scope of the invention encompasses novel compositions of matter for the inhibition of transcription. Specifically, the composition of the invention are directed to inhibiting transcription processes wherein such transcription processes are mediated by the interactions of P-TEFb and a target transcription factor. The target transcription factor may be any transcription factor that mediates a transcriptional process by interactions with P-TEFb. P-TEFb is specifically involved in the elongation step of transcription. Transcription processes, as referred to herein, may encompass the initiation, elongation, and termination of RNA synthesis from DNA sequences encoding genes that produce proteins or non-coding RNAs, gene expression of genes expressed under the control of the transcription factor, translation of proteins coded by genes expressed under the control of the transcription factor, and/or the activity or biological actions of proteins coded by genes expressed under the control of the transcription factor. Inhibiting means reducing, blocking, ablating or otherwise reducing the occurrence, magnitude or rate of a transcription process.

In a primary implementation, the therapeutic compositions of the invention encompass a multifunctional agent, wherein the agent achieves the following three functions:
recruiting the target transcription factor to the trifunctional molecule;
maintaining the CDK9 subunit of P-TEFb in an inactive state; and
inhibiting binding of the target transcription factor to P-TEFb to activate P-TEFb.
   Advantageously, the inventors of the present disclosure have developed tri-functional fusion proteins that can perform these functions in a single agent. The scope of the invention encompasses any composition of matter which can effect the three enumerated functions. The composition of matter may comprise a protein, a small molecule, a lipid, a carbohydrate, a nucleic acid, or a hybrid complex of different molecular components (e.g. peptide-nucleic acid conjugates, drug-antibody conjugates, etc.).

In a preferred implementation, the therapeutic compositions of the invention encompass a single composition of matter which achieves the three functions, however, it will be understood that the scope of the invention further encompasses the use of multiple agents in combination to achieve the three functions. In a variation of the invention, the therapeutic composition of the invention effectively inhibits the targeted transcriptional activity by achieving two functions: maintaining the CDK9 subunit of P-TEFb in an inactive state, and inhibiting binding of the target transcription factor to activate P-TEFb.

The ordering of domains disclosed herein is especially efficacious in performing the functions of the invention, however the scope of the invention extends to variations on the exemplary arrangements presented herein, wherein the fusion proteins are configured with different ordering of the elements.

The scope of the invention encompasses novel chimeric protein constructs that may be used to specifically inhibit P-TEFb-dependent transcription mediated by various transcription factors such as HIV Tat, NFkB, and cMyc in the inhibition of aberrant or undesirable transcription mediated by the action of a transcription factor with P-TEFb. In one embodiment, the chimeric construct comprises a fusion protein configured, from N to C terminus, as:

### (Transcription Factor Recruitment Domain)-(CDK9 Inhibiting Domain)-(P-TEFb Binding Domain)

wherein the Transcription Factor Recruitment Domain is an element that facilitates the interaction, including binding or other association, of the fusion protein with the target transcription factor; the CDK9 inhibiting domain maintains the CDK9 subunit of P-TEFb in a transcriptionally inactive state, and the P-TEFb binding domain competes with the target transcription factor for P-TEFb binding.

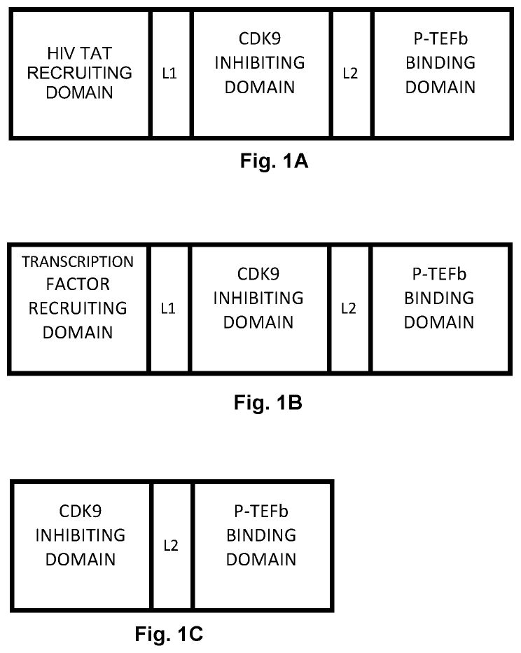

In one implementation, the fusion protein of the invention is configured as depicted in FIG. 1A, wherein a first linker sequence is present between the recruitment domain and the CDK9 inhibitory domain, and a second linker sequence is present between the inhibitory and P-TEFb-binding domains. Each element is next described in more detail.

The Transcription Factor Recruitment Domain element is an element that specifically facilitates interaction of the fusion protein with the target transcription factor, for example, promoting the binding, targeting, or recruitment of the transcription factor to the chimeric protein. The Transcription Factor Binding Domain may comprise a motif that, functionally, brings the chimeric fusion protein to the transcription factor. The element may act by binding to the transcription factor, binding to cofactors or subunits of the transcription factor transactivating complex, or binding to transcription factor binding sites on the elongation complex of a DNA or, in the case of viral infections, RNA strand.

In various embodiments, the transcription factor recruitment domain is an element that facilitates interaction with a transcription factor selected from the group consisting of HIV Tat, NFkB, cMyc, MyoD, STAT-family transcription factors, Class II transactivator (CIITA), Autoimmune Regulator (AIRE), Eleven Nineteen Leukemia (ENL), AF4 proteins implicated in acute leukemia, estrogen receptors, androgen receptors, MEF2, and HTLV1 Tax.

In one aspect, the scope of the invention encompasses novel chimeric fusion proteins and methods of using them for the inhibition of HIV transcription. In one embodiment, the chimeric construct comprises a fusion protein configured, from N to C terminus, as:

(TAR/Tat Interacting Domain)-(CDK9 inhibiting domain)-(P-TEFb binding domain)
wherein the TAR/Tat inhibitor binds the HIV-Tar complex with target RNA and disrupts TAR/Tat complex formation. In one embodiment, the TAR/Tat inhibitor comprises an arginine rich motif (ARM). The ARM domain may comprise any basic or arginine rich protein domain which binds to TAR and disrupts Tat/P-TEFb cooperative binding to TAR. In one embodiment, the TBF of the fusion protein comprises an ARM the human HEXIM1 protein. In one embodiment, the human HEXIM1 ARM comprises amino acids 150-177 of the human HEXIM1 protein (SEQ ID NO: 1), or a variant thereof.

In another embodiment, the ARM comprises an HIV-1 Tat ARM, for example, amino acids 49-57 of the HIV-1 Tat protein (SEQ ID NO: 2), or a variant thereof.

Regarding the second element of the fusion protein, the inhibitory domain, this domain inhibits the transcriptional activity of CDK9. In one embodiment, this element comprises the HEXIM1 P-TEFb inhibitory domain, which achieves full inhibition of P-TEFb CDK9 kinase activity, including, for example inhibition of phosphorylation of transcriptional inhibitory complexes NELF and DSIF or of the RNAPII C-terminal domain. For example, the ID domain of the chimera may comprise amino acids 188-220 of HEXIM1 (SEQ ID NO: 3), or a variant thereof.

Regarding the P-TEFb-binding domain, this domain may encompass any peptide or other composition which binds P-TEFb with high affinity, for example, binding to CycT1 or CycT2. In HIV infection, one function of the Tat protein is to displace P-TEFb from the inhibitory HEXIM1-7SK snRNP complex by competitive binding. This is achieved by the Tat's AD domain which has high affinity for CycT1. In one embodiment, the P-TEFb-binding domain comprises the P-TEFb binding domain of HIV-1 Tat, for example, amino acids 1-48 of HIV-1 Tat (SEQ ID NO: 4), or a variant thereof.

The fusion proteins of the invention may optionally comprise a spacer or linker sequence disposed between the elements. In one embodiment, the chimeric proteins of the invention comprise a spacer sequence moieties disposed between transcription factor recruitment element and the CDK9 Inhibitor element. In one embodiment, the chimeric proteins of the invention comprise a spacer sequence between the CDK9 inhibitor domain and the P-TEFb binding domain. In some embodiments, both the first and second spacer are present. In some embodiments, one or both of the spacer sequences is omitted. The space sequence length may comprise any amino acid sequence, for example, a sequence having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, a value of 30 to 40, a value of 40 to 50, a value of 50 to 60, a value of 60 to 70 a value of 70 to 80, a value of 80-90, a value of 90-100, or a value of over 100 amino acids. Preferably, the linker will be a biologically inactive polypeptide and will be flexible. Exemplary linker sequences comprise glycine and/or serine rich sequences, for example, sequences comprising at least 50% glycine and/or serine, at least 75% glycine and/or serine, or at least 90% glycine and/or serine. In one embodiment, the linker comprises one or more linker sequences of GGGGS (SEQ ID NO: 5), for example, 1, 2, 3, 4, 5, 6, 7, 8, 9, 10, 11, 12, 13, 14, 15, 16, 17, 18, 19, or 20 repeats of GGGGS.

In one aspect, the scope of the invention encompasses various combinations of the afore-described elements for the inhibition of HIV replication. In one embodiment, the fusion protein chimera of the invention is HT1 (SEQ ID NO: 6), or a variant thereof, made up of the HEXIM1 ARM, the HEXIM1 CDK9 inhibitory domain, and the TAT P-TEFb binding domain.

In another embodiment, the fusion protein chimera of the invention is the THT construct (SEQ ID NO: 7), or a variant thereof. THT utilizes the ARM of the HIV1 Tat protein as the recruitment domain, the HEXIM 1 CDK9 inhibitory domain, and the TAT P-TEFb-binding domain, and achieves powerful inhibition of HIV transcription.

The inventors of the present disclosure have advantageously discovered that P-TEFb-mediated transcriptional processes can be inhibited by a chimeric protein lacking the transcription factor recruitment element. In this implementation, the first element and linker sequence of the fusion protein construct are omitted. In one embodiment, the fusion protein chimera of the invention comprises a HEXIM 1 CDK9 inhibitory domain and a TAT P-TEFb-binding domain, as in HT3, SEQ ID NO: 8.

It will be understood that the scope of the invention encompasses variants of the enumerated sequences referenced and disclosed herein. For example, variants may include truncations, deletions, insertions, or replacements of the enumerated amino acid or nucleic acid sequences sequences, including conservative or radical replacements, and substitutions with non-natural amino acids and amino acid analogs or nucleotides, as the case may be. Variants of an enumerated amino acid sequence may have at least 70%, at least 75%, at least 80%, at least 85%, at least 90%, at least 95% or at least 99% identity to enumerated sequences, wherein variants of an enumerated nucleic acid sequence may comprise, in various embodiments, at least 70%, at least 75%, at least 80%, at least 85%, at least 90%, at least 95% or at least 99% nucleic acid identity or homology to the enumerated sequence.

The chimeric fusion proteins of the invention may further comprise one or more additional elements, such as additional polypeptides, functional polypeptides, or peptide modifications, for example to conjugate chemical or biological entities. Additional elements may include therapeutic moieties, stabilizing elements, targeting elements, or elements to reduce immunogenic responses. In one embodiment, the additional elements comprise cell secretion signal and/or a cell penetration signal that enable the inhibitor to be secreted from the cells that produce it and incorporated by bystander cells, as known in the art. The one or more additional functional molecules may be incorporated at the C terminus of the fusion protein, at the N-terminus of the fusion protein, or may be incorporated into the first and/or second linker sequence.

Nucleic Acid Constructs and Cells.

The scope of the invention further encompasses nucleic acid constructs coding for the chimeric proteins of the invention, including DNA, RNA, and other nucleic acid types. The nucleic acid construct may comprise a plasmid, expression vector, or transformed cell genome. The scope of the invention further encompasses transformed animals that express the chimeric proteins of the invention in some or all cells of the organism, including test animals and human subjects. In one embodiment, the scope of the invention encompasses an expression vector, wherein the expression vector codes for a protein comprising a fusion protein of the invention, the expression vector being configured for delivery to target cells and being under the control of a promoter that enables expression of the fusion protein by the target cells.

In one embodiment, the scope of the invention encompasses a nucleic acid coding for the HT1 fusion protein, for example, SEQ ID NO: 10 or a variant thereof, for example, a variant comprising at least 95% sequence identity or homology to SEQ ID NO: 10. In one embodiment, the scope of the invention encompasses a nucleic acid coding for the HT3 fusion protein, for example, SEQ ID NO: 11 or a variant thereof, for example, a variant comprising at least 95% sequence identity or homology to SEQ ID NO: 11. In one embodiment, the scope of the invention encompasses a nucleic acid coding for the THT protein, for example, SEQ ID NO: 12 or a variant thereof, for example, a variant comprising at least 95% sequence identity or homology to SEQ ID NO: 12.

Methods of Treatment.

The scope of the invention encompasses the treatment of subjects by the administration of the therapeutic compositions of the invention to subjects in need of treatment therefor. In one embodiment, the subject is a subject suffering from a condition wherein transcriptional activity is a factor in the development, progression, pathology, symptoms, or persistence of a disease or condition and wherein the transcriptional activity is mediated by P-TEFb interactions with a target transcription factor. In one embodiment the target transcription factor is selected from the group consisting of HIV Tat, NFkB, cMyc, MyoD, STAT-family transcription factors, Class II transactivator (CIITA), Autoimmune Regulator (AIRE), Eleven Nineteen Leukemia (ENL), AF4 proteins implicated in acute leukemia, estrogen receptors, androgen receptors, MEF2, and HTLV1 Tax. In one embodiment, the condition is a hypertrophic or hyperproliferative disease. In one embodiment, the condition is cancer or other proliferative disease. In one embodiment, the condition is an inflammatory condition, such as airway inflammation. In one embodiment, the condition is myocardial hypertrophy. In one embodiment, condition is a viral infection, for example, a retroviral infection, for example, HIV infection, for example, infection by HIV-1 or HIV-2. The subject may be any animal, for example, a human subject, for example, a human patient, or a non-human veterinary subject or test animal.

Advantageously, the engineered peptides of the invention are small in size. In one implementation, the fusion proteins of the invention are administered to target cells. Such peptides may be modified with cell-penetrating agents, nuclear localization signals, and or other carriers and/or targeting moieties. The therapeutic proteins of the invention may be administered intravenously, or by other modalities known in the art, for example by methods of administering protein therapeutics orally, topically, transdermally, subcutanouesly etc.

The therapeutic molecules of the invention may be administered in pharmaceutically effective amount, for sufficient to achieve cellular concentrations in the range of nanomolar to millimolar concentrations.

In one embodiment, the therapeutic molecules are administered by gene therapy, wherein cells of the host (e.g. human patient) are transformed to express the chimeric proteins by a gene therapy construct encoding a chimeric protein of the invention. Such gene therapy constructs may be delivered by means known in the art. Nucleic acid construct delivery to target cells, for example, endothelial cells of the artery, may be achieved by any means known in the art. For example, delivery may be achieved by viral gene vectors (e.g. lentivirus or adenovirus), electroporation, biolistic delivery systems, microinjection, ultrasound, hydrodynamic delivery, liposomal delivery, polymeric or protein-based cationic agents (e.g. polyethylene imine, polylysine), intraject systems, and DNA-delivery dendrimers. Target cells may include, for example, cancerous cells, epithelial cells, endothelial cells, immune cells, and other cell types wherein P-TEFb mediated action of the target transcription factor is targeted. For example, the CD4+, dendritic cells, and macrophages, and their precursors, such as monocytes and bone marrow stem cells. In one strategy, producer cells are transformed to produce the peptides of the invention, wherein the peptides are modified with secretion and uptake signaling peptides that facilitate their secretion by producer cells an uptake by target cells.

In one embodiment, the subject is in need of treatment for HIV infection. The subject may be a human subject at risk of contracting an HIV infection, may be a subject with a latent HIV infection, or may be a subject with AIDS. The HIV infection may be an HIV-1 infection or an HIV-2 infection. The treatment may comprise a preventative treatment, or a therapeutic treatment. The therapeutic treatment may achieve any reduction in HIV replication, or may achieve any therapeutic effect including: inhibition of viral escape, inhibited expression of integrated provirus, inhibition of viral budding, inhibition of viral mutation, suppression of viral replication following cessation of cART, prevention of sporadic reactivation of integrated HIV, reduction in HIV persistence and reservoir replenishment, reduction in HIV associated chronic inflammation, promotion of HIV latency, and cure of HIV infection. The methods and compositions of the invention may be applied in combination with other therapies, including therapies that block other steps of the HIV infection process such as viral entry, reverse transcription, integration and maturation steps, for example, as treated by CART.

### Exemplary Embodiments

The scope of the invention encompasses a fusion protein, for use in a method of inhibiting transcription mediated by P-TEFb and a selected target transcription factor;

wherein the fusion protein comprises
a first element comprising a transcription factor recruitment element which facilitates interaction between the fusion protein and the selected target transcription factor;
a first linker sequence;
a second element comprising a CDK9 inhibiting domain that maintains the CDK9 subunit of P-TEFb in an inactive state;
a second linker sequence; and
a third element comprising a P-TEFb-binding domain that competes with the target transcription factor for P-TEFb binding;
wherein the target transcription factor may be any transcription factor that mediates a transcriptional activity by interactions with P-TEFb, in some embodiments the transcription factor being selected from the group consisting of HIV Tat, in one embodiment, HIV-1 Tat, NFkB, cMyc, MyoD, STAT-family transcription factors, Class II transactivator (CIITA), Autoimmune Regulator (AIRE), Eleven Nineteen Leukemia (ENL), AF4 proteins implicated in acute leukemia, estrogen receptors, androgen receptors, MEF2, and HTLV1 Tax.

In some embodiments, the second element comprises the CDK9 inhibiting element of HEXIM1, in one embodiment, amino acids 188-220 (SEQ ID NO: 3) or a variant of SEQ ID NO: 3, in one embodiment the variant having at least 95% sequence identity to SEQ ID NO: 3, and the third element comprise the P-TEFb-binding domain of HIV-1 TAT, in one embodiment, the amino acids 1-48 of HIV-1 Tat (SEQ ID NO: 4) or a variant of SEQ ID NO: 4, in one embodiment the variant having at least 95% sequence identity to SEQ ID NO: 4.

In some embodiments, the first linker sequence between the first and second elements and/or the second linker sequence between the second and third elements is omitted. In some embodiments, the first linker sequence and/or the second linker sequence comprises a flexible linker sequence. In some embodiments, the first linker sequence and/or the second linker sequence comprises a serine and/or glycine rich amino acid sequence, in some implementations, comprising at least 50%, at least 60%, 70%, at least 80%, or at least 90% serine and/or glycine residues. In certain embodiments the first linker sequence and/or the second linker sequence comprises one or more repeats, of SEQ ID NO: 5, in some embodiments the number of repeats being 1-20 repeats, in certain embodiments being 1, 2, 3, 4, 5, 6, 7, 8, 9, 10, 11, 12, 13, 14, 15, 16, 17, 18, 19, 20, or more than 20 repeats. In certain embodiments, the first linker sequence and/or the second linker sequence comprises 1-100 amino acids, in some embodiments being 1-10 amino acids, 10-20 amino acids, 20-30 amino acids, 30-40 amino acids, 40-50 amino acids, 50-60 amino acids, 60-70 amino acids, 70-80 amino acids, 80-90 amino acids, 90-100 amino acids, or more than 100 amino acids in length.

In some implementations, the scope of the invention encompasses a fusion protein for use in a method selected from the inhibition of HIV replication mediated by P-TEFb, the prevention or treatment of HIV infection, the prevention of reactivation of integrated HIV, or the prevention of an HIV Tat-mediated transcriptional process, the fusion protein comprising: a first element comprising a transcription factor recruitment element which facilitates interaction between the fusion protein and HIV Tat, in one embodiment being HIV-1 Tat; a first linker sequence; a second element comprising a CDK9 inhibiting domain that maintains the CDK9 subunit of P-TEFb in an inactive state; a second linker sequence; and a third element comprising a P-TEFb-binding domain that competes with the target transcription factor for P-TEFb binding; wherein, in some embodiments, the transcription factor recruitment element which facilitates interaction between the fusion protein and HIV Tat is an arginine-rich domain, in some embodiments the Tat recruiting element comprises the HEXIM1 arginine rich motif, in some embodiments the HEXIM 1 arginine rich motif comprising SEQ ID NO: 1 or a variant thereof, in one embodiment the variant comprising at least 95% sequence identity to SEQ ID NO: 1; in some embodiments the Tat recruiting element comprises the HIV-1 Tat arginine rich motif, in some embodiments the HIV-1 Tat arginine rich motif comprising SEQ ID NO: 2 or a variant thereof, in one embodiment the variant comprising at least 95% sequence identity to SEQ ID NO: 2; wherein in some embodiments the CDK9 inhibiting element comprises the CDK9 inhibiting element of HEXIM1, in one embodiment, amino acids 188-220 of HEXIM1 (SEQ ID NO: 3) or a variant of SEQ ID NO: 3, in one embodiment the variant comprising at least 95% sequence identity to SEQ ID NO: 3, and wherein, in some embodiments, the P-TEFb-binding element comprises the P-TEFb-binding domain of HIV-1 TAT, for example amino acids 1-48 of HIV-1 Tat (SEQ ID NO: 4) or a variant SEQ ID NO: 4, in one embodiment the variant comprising at least 95% sequence identity to SEQ ID NO: 4. In some embodiments of the fusion protein for use in a method of inhibiting HIV transcription mediated by P-TEFb the linker sequence comprises a flexible linker sequence; in some embodiments, the first linker sequence and/or the second linker sequence comprises a serine and/or glycine rich amino acid sequence, for example comprising at least 50%, at least 60%, 70%, at least 80%, or at least 90% serine and/or glycine residues. In certain embodiments the first linker sequence and/or the second linker sequence comprises one or more repeats of SEQ ID NO: 5, in some embodiments the number of repeats being 1-20 repeats, in certain embodiments being 1, 2, 3, 4, 5, 6, 7, 8, 9, 10, 11, 12, 13, 14, 15, 16, 17, 18, 19, 20, or more than 20 repeats; in certain embodiments, the first linker sequence and/or the second linker sequence comprises 1-100 amino acids, in some embodiments being 1-10 amino acids, 10-20 amino acids, 20-30 amino acids, 30-40 amino acids, 40-50 amino acids, 50-60 amino acids, 60-70 amino acids, 70-80 amino acids, 80-90 amino acids, 90-100 amino acids, or more than 100 amino acids in length.

In certain embodiments, the fusion protein for use in a method selected from the inhibition of HIV replication mediated by P-TEFb, the prevention or treatment of HIV infection, the prevention of reactivation of integrated HIV, or the prevention of an HIV Tat-mediated transcriptional process, the fusion protein comprises SEQ ID NO: 6 or a variant thereof, in one embodiment the variant comprising at least 95% sequence identity to SEQ ID NO: 6, or SEQ ID NO: 7 or a variant thereof, in one embodiment the variant comprising at least 95% sequence identity to SEQ ID NO: 7.

In certain embodiments, the scope of the invention encompasses a nucleic acid construct which codes for a fusion protein, wherein the fusion protein comprises a transcription factor recruitment element which facilitates interaction between the fusion protein and the selected target transcription factor; a first, optional linker sequence; a second element comprising a CDK9 inhibiting domain that maintains the CDK9 subunit of P-TEFb in an inactive state; a second, optional, linker sequence; and a third element comprising a P-TEFb-binding domain that competes with the target transcription factor for P-TEFb binding; wherein the target transcription factor may be any transcription factor that mediates a transcriptional activity by interactions with P-TEFb, in some embodiments the transcription factor being selected from the group consisting of HIV Tat, NFkB, cMyc, MyoD, STAT-family transcription factors, Class II transactivator (CIITA), Autoimmune Regulator (AIRE), Eleven Nineteen Leukemia (ENL), AF4 proteins implicated in acute leukemia, estrogen receptors, androgen receptors, MEF2, and HTLV1 Tax; wherein in certain embodiments, the recruitment element is omitted; wherein, in certain embodiments, the target transcription factor is Tat and recruitment element facilitates interaction between the fusion protein and HIV Tat; wherein in certain embodiments, the recruitment factor is an arginine-rich domain; wherein, in some embodiments the Tat recruiting element comprises the HEXIM1 arginine rich motif, in some embodiments the HEXIM 1 arginine rich motif comprising SEQ ID NO: 1 or a variant thereof, in one embodiment the variant comprising at least 95% sequence identity to SEQ ID NO: 1; in some embodiments the Tat recruiting element comprises the HIV-1 Tat arginine rich motif, in some embodiments the HIV-1 Tat arginine rich motif comprising SEQ ID NO: 2 or a variant thereof, in one embodiment the variant comprising at least 95% sequence identity to SEQ ID NO: 2; wherein in some embodiments the CDK9 inhibiting element comprises the CDK9 inhibiting element of HEXIM1, in one embodiment amino acids 188-220 of HEXIM (SEQ ID NO: 3), or a variant of SEQ ID NO: 3, in one embodiment the variant comprising at least 95% sequence identity to SEQ ID NO: 3, and wherein, in some embodiments, the P-TEFb-binding element comprises the P-TEFb-binding domain of HIV-1 TAT, for example amino acids 1-48 of HIV-1 Tat (SEQ ID NO: 4) or a variant of SEQ ID NO: 4, in one embodiment the variant comprising at least 95% sequence identity to SEQ ID NO: 4; wherein in certain embodiments the fusion protein comprises SEQ ID NO: 6, or a in one embodiment, a variant comprising 95% sequence identity to SEQ ID NO: 6, or SEQ ID NO: 7 or a variant thereof, in one embodiment, a variant comprising 95% sequence identity to SEQ ID NO: 7; wherein the nucleic acid construct coding for the fusion protein may comprise any of a plasmid, an expression vector, a transformation vector or cassette, or the transformed genome of an organism; wherein, in certain embodiments, the nucleic acid construct is a gene expression vector configured for the transformation of target cells and the expression of the fusion protein by or within the target cells.

In certain embodiments, the invention encompasses a method of treating a subject in need of treatment for a condition wherein a transcriptional process is implicated in the condition, wherein the transcriptional process is mediated by P-TEFb interactions with a selected target transcription factor, wherein in various embodiments, the condition is mediated by P-TEFb interaction with a target transcription factor; wherein in certain embodiments, the target transcription factor may be being selected from the group consisting of HIV Tat, NFkB, cMyc, MyoD, STAT-family transcription factors, Class II transactivator (CIITA), Autoimmune Regulator (AIRE), Eleven Nineteen Leukemia (ENL), AF4 proteins implicated in acute leukemia, estrogen receptors, androgen receptors, MEF2, and HTLV1 Tax; wherein in certain embodiments, the condition is selected from the group consisting of HIV infection, cancer, cardiac hypertrophy, or an inflammatory condition; the method of treatment comprising the administration of a pharmaceutically effective amount of the fusion protein, wherein the fusion protein comprises a transcription factor recruitment element which facilitates interaction between the fusion protein and the selected target transcription factor; a first, optional linker sequence; a second element comprising a CDK9 inhibiting domain that maintains the CDK9 subunit of P-TEFb in an inactive state; a second, optional, linker sequence; and a third element comprising a P-TEFb-binding domain that competes with the target transcription factor for P-TEFb binding; wherein the target transcription factor may be any transcription factor that mediates a transcriptional activity by interactions with P-TEFb, in some embodiments the transcription factor being selected from the group consisting of HIV Tat, in one embodiment being HIV-1 Tat, NFkB, cMyc, MyoD, STAT-family transcription factors, Class II transactivator (CIITA), Autoimmune Regulator (AIRE), Eleven Nineteen Leukemia (ENL), AF4 proteins implicated in acute leukemia, estrogen receptors, androgen receptors, MEF2, and HTLV1 Tax; wherein in certain embodiments, the recruitment element is omitted; wherein; wherein in some embodiments the CDK9 inhibiting element comprises the CDK9 inhibiting element of HEXIM1, in one embodiment being amino acids 188-220 of HEXIM 1 (SEQ ID NO: 3) or a variant SEQ ID NO: 3, in one embodiment, a variant comprising 95% sequence identity to SEQ ID NO: 3, and in some embodiments, the P-TEFb-binding element comprises the P-TEFb-binding domain of HIV-1 TAT, in one embodiment being amino acids 1-48 of HIV-1 Tat (SEQ ID NO: 4) or a variant of SEQ ID NO: 4, in one embodiment, a variant comprising 95% sequence identity to SEQ ID NO: 4. In some embodiments, the method of treatment is directed to the prevention or treatment of an HIV infection, in one embodiment being an HIV-1 infection, wherein in some embodiments the treatment is applied to effect a therapeutic outcome, in some embodiments being selected from the group consisting of preventing HIV infection, treating a latent HIV infection, treating a subject with AIDS, a reduction in HIV replication, inhibition of viral escape, inhibited expression of integrated provirus, inhibition of viral budding, inhibition of viral mutation, suppression of viral replication following cessation of cART, prevention of sporadic reactivation of integrated HIV, reduction in HIV persistence and reservoir replenishment, reduction in HIV associated chronic inflammation, promotion of HIV latency, and cure of HIV infection, and wherein, in certain embodiments, the target transcription factor is Tat and recruitment element facilitates interaction between the fusion protein and HIV Tat; wherein in certain embodiments, the recruitment factor is an arginine-rich domain; wherein, in some embodiments the Tat recruiting element comprises the HEXIM1 arginine rich motif, in some embodiments the HEXIM 1 arginine rich motif comprising SEQ ID NO: 1 or a variant thereof, in one embodiment, a variant comprising 95% sequence identity to SEQ ID NO: 1; in some embodiments the Tat recruiting element comprises the HIV-1 Tat arginine rich motif, in some embodiments the HIV-1 Tat arginine rich motif comprising SEQ ID NO: 2 or a variant thereof, in one embodiment, a variant comprising 95% sequence identity to SEQ ID NO: 2; wherein in certain embodiments the fusion protein comprises SEQ ID NO: 6, or a variant thereof, in one embodiment, a variant comprising 95% sequence identity to SEQ ID NO: 6, or SEQ ID NO: 7 or a variant thereof, in one embodiment, a variant comprising 95% sequence identity to SEQ ID NO: 7; wherein in some embodiments the administration of a pharmaceutically effective amount of the fusion protein is achieved by administration of the protein to the subject; and wherein in some embodiments, the administration of a pharmaceutically effective amount of the fusion protein comprises the administration to the subject of a nucleic acid construct coding for the fusion protein, wherein the nucleic acid construct is a gene expression vector configured for the transformation of target cells and the expression of the fusion protein by or within the target cells.

In some embodiments, the scope of the invention encompasses a fusion protein for use in a method of inhibiting transcription mediated by P-TEFb and any selected target transcription factor, wherein the target transcription factor may be a transcription factor that mediates a transcriptional activity by interactions with P-TEFb, in certain embodiments the transcription factor being HIV Tat, in one embodiment being HIV-1 Tat, NFkB, cMyc, MyoD, STAT-family transcription factors, Class II transactivator (CIITA), Autoimmune Regulator (AIRE), Eleven Nineteen Leukemia (ENL), AF4 proteins implicated in acute leukemia, estrogen receptors, androgen receptors, MEF2, and HTLV1 Tax; wherein the fusion protein comprises a an element comprising a CDK9 inhibiting domain that maintains the CDK9 subunit of P-TEFb in an inactive state;

an optional intervening linker sequence; and an element comprising a P-TEFb-binding domain that competes with the target transcription factor for P-TEFb binding; and optionally comprises a linker sequence disposed between the elements, in some embodiments the linker sequence comprising a flexible linker sequence. In some embodiments, the first linker sequence and/or the second linker sequence comprises a serine and/or glycine rich amino acid sequence, in some embodiments comprising at least 50%, at least 60%, 70%, at least 80%, or at least 90% serine and/or glycine residues. In certain embodiments the first linker sequence and/or the second linker sequence comprises one or more repeats, of SEQ ID NO: 5, in some embodiments the number of repeats being 1-20 repeats, in certain embodiments being 1, 2, 3, 4, 5, 6, 7, 8, 9, 10, 11, 12, 13, 14, 15, 16, 17, 18, 19, 20, or more than 20 repeats. In certain embodiments, the first linker sequence and/or the second linker sequence comprises 1-100 amino acids, in some embodiments being 1-10 amino acids, 10-20 amino acids, 20-30 amino acids, 30-40 amino acids, 40-50 amino acids, 50-60 amino acids, 60-70 amino acids, 70-80 amino acids, 80-90 amino acids, 90-100 amino acids, or more than 100 amino acids in length. In some embodiments, the CDK9 inhibiting element comprises the CDK9 inhibiting element of HEXIM1, in one embodiment being amino acids 188-220 of HEXIM1 (SEQ ID NO: 3) or a variant of SEQ ID NO: 3, in one embodiment, a variant comprising 95% sequence identity to SEQ ID NO: 3, and the P-TEFb binding element comprises the P-TEFb-binding domain of HIV-1 TAT, in one embodiment being amino acids 1-48 of HIV-1 Tat (SEQ ID NO: 4) or a variant of SEQ ID NO: 4, in one embodiment, a variant comprising 95% sequence identity to SEQ ID NO: 4. In some embodiments, the fusion protein comprises SEQ ID NO: 8 or a variant thereof, in one embodiment, a variant comprising 95% sequence identity to SEQ ID NO: 8.

In some embodiments, the scope of the invention encompasses a method of using a fusion protein as described herein in the manufacture of a medicament for the treatment of a condition, wherein the medicament is directed to the treatment of a condition by inhibiting a transcriptional activity of a target transcription factor mediated by the interaction of the transcription factor with P-TEFb; in various embodiments the target transcription factor may be selected from the group consisting of of HIV Tat, for example, HIV Tat, NFkB, cMyc, MyoD, STAT-family transcription factors, Class II transactivator (CIITA), Autoimmune Regulator (AIRE), Eleven Nineteen Leukemia (ENL), AF4 proteins implicated in acute leukemia, estrogen receptors, androgen receptors, MEF2, and HTLV1 Tax; wherein in certain embodiments, the condition is selected from the group consisting of HIV infection, cancer, cardiac hypertrophy, or an inflammatory condition.

### EXAMPLES

### Example 1. HEXIM1-Tat Chimera Inhibits HIV-1 Replication

Treatment with combination antiretroviral therapy (cART) leads to efficient suppression of HIV replication, but HIV persistence in latently infected cells remains an obstacle to cure. Even under cART, residual HIV replication can arise and ultimately lead to the emergence of resistance mutations and viral escape. Targeting diverse steps of the viral cycle is the most efficient way to prevent viral escape. Currently, viral entry, reverse transcription, integration and maturation steps have been targeted by cART. However, no efficient transcription inhibitor is clinically available, though multiple strategies—such as TAR decoys or dominant-negative Tat—have been explored to prevent expression of the integrated provirus.

Blocking transcription would not only add another therapeutic target, but also prevent sporadic reactivation of integrated HIV that may contribute to HIV persistence, reservoir replenishment and chronic inflammation. Suppressing residual HIV transcription is also the goal of the emerging block and lock HIV cure strategies, which aim at deepening HIV latency so that integrated proviruses remains definitively locked in the infected cells.

HIV expression is dependent on the viral transactivator Tat, which brings the cellular positive transcription elongation factor B (P-TEFb) to the HIV promoter. P-TEFb is comprised of cyclin T1 (CycT1) and cyclin-dependent kinase 9 (CDK9), and is required for transcription elongation, both for HIV and host gene expression. In cells, most of P-TEFb is sequestered in the 7SK small nuclear ribonucleoprotein complex (7SK snRNP), which includes a non-coding 7SK snRNA and proteins HEXIM1, LARP7 and MePCE. In 7SK snRNP, the transcriptional regulator HEXIM1 binds to the 7SK snRNA through a RNA-binding arginine rich motif (ARM, residues 150-162), and to P-TEFb through its CycT1 binding domain (TBD, residues 250-359) and its central inhibitory domain (ID, residues 200-211). This ID includes a PYNT motif (²⁰²Pro-²⁰³Tyr-²⁰⁴Asn-²⁰⁵Thr), which masks CDK9's substrate-binding site and is critical for its inactivation. Importantly, HEXIM1's TBD acts synergistically with ID on Cdk9 inhibition.

Without recruitment of active P-TEFb to the HIV promoter, RNA polymerase II (RNAPII) is stalled after having only transcribed the short transactivation response element (TAR) RNA, located at the 5′ end of all viral transcripts. The Tat activation domain (AD, residues 1-48) binds CycT1, while its central ARM binds to 7SK RNA, thus displacing P-TEFb from the 7SK snRNP and releasing it from HEXIM1 inhibition. Tat and CycT1 also form a cooperative binding surface for TAR, where the central ARM region of Tat (residues 51-57) binds to the bulge region of TAR and the Tat-TAR recognition motif of CycT1 binds to the central loop of TAR. These interactions allow P-TEFb to be recruited to the RNAPII early elongation complex that is stalled at the HIV transcription start site. There, CDK9 phosphorylates transcriptional inhibitory complexes NELF and DSIF as well as RNAPII C-terminal domain (CTD), resulting in stimulation of transcription elongation.

Interestingly, Tat/TAR/P-TEFb interaction structurally mimics that of HEXIM1/7SK/P-TEFb, and the amino-acid sequences of Tat and HEXIM1 ARMs are almost identical. Since the former is a strong transcription activator for HIV, while the latter is a potent inhibitor of P-TEFb, we sought to create a HIV-specific transcription inhibitor by taking advantage of the structure similarities of these two complexes. We designed chimeras that derive from critical functional domains of Tat and HEXIM1, by combining the P-TEFb binding N-terminal domain of Tat to the acidic and/or central basic domains of HEXIM1 that respectively inhibit P-TEFb and bind RNA. A small HEXIM1-Tat chimera, HT1, inhibited HIV transcription by preventing the recruitment of active P-TEFb to TAR, with only little off-target effects on cellular genes. This proof of concept study demonstrates the feasibility of designing highly specific transcriptional inhibitor chimeras.

Results

A HEXIM1/Tat Chimera Inhibits Transcription from the HIV Promoter

We screened a collection of chimeras derived from the ARM and/or ID of HEXIM1 fused to the AD of Tat (Tat1-48). These chimeras include: HT1 (SEQ ID NO: 6) with both HEXIM1 domains of interest (Hex150-220), HT2 (SEQ ID NO: 9) with only HEXIM1 ARM (Hex150-177) fused to the AD of Tat and HT3 (SEQ ID NO: 8) with only HEXIM1 ID (Hex178-220) fused to the AD of Tat.

Luciferase (Luc) reporter assays were performed to titrate the potency of each chimera to inhibit Tat-dependent gene expression from the HIV promoter. Effector plasmids included pHT1-3 and pTat, which respectively expressed a Myc-epitope tag chimera (m:HT1-3) and a Flag-epitope tagged Tat (f:Tat). pHT1, pHT2 or pHT3 was transiently co-transfected in 293T cells with pTat and a Luc reporter gene under the control of the HIV promoter (P-HIV, expressed from the plasmid pLTR-Luc). HT2, which does not include HEXIM1 ID, failed to inhibit Luc expression from the HIV promoter, even when the ratio of pHT2 to pTat was 2:1 HT3, which included the ID, induced up to a 2-fold decrease in Luc expression from the HIV promoter at the 2:1 ratio. Finally, HT1 lead to a 4-fold decrease in Luc expression at a 2:1 ratio. Of note, including other domains from HEXIM1 or Tat did not improve the potency of the chimeras. The stronger inhibitory effect by HT1 when compared to that by HT3 suggested that HEXIM1 ARM also contributes to the inhibition of Tat-induced Luc expression from the HIV promoter. Importantly, mutating the PYNT motif, which is critical for HEXIM1's Cdk9 inhibition, abolished the inhibitory effect of HT1 suggesting that HT1's inhibitory effect on HIV transcription is mediated by Cdk9 inhibition. Adding different lengths of flexible peptide linkers (GGGGS, SEQ ID NO: 5) slightly, but insignificantly improved the inhibitory effects of HT1. Also, reversing the order of peptide motif also abolished HT1's inhibitory effect, indicating that precise spatial arrangement of these motifs is required. Similar results were obtained when co-transfecting pHT1-3 and pTat in NIH1 cells, which stably carry an LTR-Luc reporter gene. Thus, we selected HT1 for further investigation.

Levels of protein expression from pHT1 and pTat in 293T cells were confirmed by western blotting and suggested that the expression of Tat may be slightly reduced upon co-transfection of pHT1. To rule out any bias in the inhibitory titration of HT1, we thus used pNL43 ΔenvLuc, a defective HIV molecular clone from which Tat is expressed from the HIV promoter and not from a separate plasmid. In this model, transient expression of HT1 led to a 3-fold decrease in Luc expression. Taken together, these results suggest that HT1 potently inhibits gene expression from the HIV promoter.

HT1 Prevents Tat from Bringing Active P-TEFb to TAR.

We next investigated the mechanisms by which HT1 can prevent HIV gene expression. Since both Tat AD and HEXIM1 ID interact with P-TEFb, we performed a series of co-immunoprecipitations to determine whether HT1 interacted with P-TEFb and competed with Tat for P-TEFb binding. m:HT1 and f:Tat were transiently expressed in 293T cells, and both bound to CycT1 as demonstrated using anti-Myc or anti-Flag antibodies (Abs) for co-immunoprecipitation. Moreover, when co-expressing a fixed amount of f:Tat and increasing amounts of m:HT1, the amounts of CycT1 co-immunoprecipitating with f:Tat decreased, suggesting a competition between HT1 and Tat for P-TEFb binding.

Next we investigated whether HT1, which contains HEXIM1 ID, inhibited the kinase activity of CDK9. m:HT1 was transiently expressed in 293T cells and immunoprecipitated using anti-Myc Ab. Co-immunoprecipitated P-TEFb was subjected to an in vitro kinase assay with ATP and recombinant GST-CTD proteins as a substrate. Similarly, m:Tat was expressed to co-immunoprecipitate P-TEFb as a positive control. Immunoprecipitated CDK9, and phosphorylated GST-CTD (CTD-P) were detected by WB using anti-CDK9 and anti-Ser2P Abs, respectively. A larger amount of CDK9 was co-immunoprecipitated by HT1 than by Tat, while more CTD-P was detected with Tat than with HT1. Relative kinase activity associated with HT1 and Tat was calculated by CTD-P band intensity normalized with CDK9, which revealed that the kinase activity of P-TEFb was decreased 3.1 fold when bound to HT1, compared to control. This suggests that once bound to P-TEFb, HT1 can inhibit the kinase activity of CDK9. Consistently, addition of another non-inhibitory P-TEFb-binding motif (PID) from Brd4 to HT1 decreased the ability to inhibit HIV transcription.

Finally, since HEXIM1 ARM resembles the TAR-binding domain from Tat, we investigated whether HT1 could bind to HIV TAR. m:HT1 or m:Tat was co-expressed in 293T cells with TAR RNA, expressed under RNA Polymerase III dependent H1 promoter, and immunoprecipitated using anti-Myc Ab or IgG as a control. TAR RNAs co-immunoprecipitated with HT1 or Tat were quantified by RT-qPCR analysis. Both HT1 and Tat immunoprecipitated TAR, though m:Tat immunoprecipitated 2.54 fold more TAR than m:HT1. Since the level of m:Tat expressed in cell lysates was higher than that of m:HT1, we normalized the amount of immunoprecipitated TAR by the protein levels of m:HT1 and m:Tat, which suggested that similar amounts of m:HT1 bound at least as much TAR as m:Tat. m:HT3, which lacked the ARM domain from HEXIM1, failed to bind TAR. To test whether HT1 affects Tat-TAR interactions, increasing amounts of m:HT1 were also co-expressed with a fixed amount of f:Tat and TAR. RNA immunoprecipitation assays were then performed using anti-Flag Ab followed by TAR RT-qPCR, and indicated that the amounts of TAR RNA co-immunoprecipitated with f:Tat decreased progressively when expression of m:HT1 increased. WB confirmed that the amounts of HT1 used for this assay did not impact on f:Tat expression, suggesting that the decrease in co-immunoprecipitated TAR was due to HT1 competing with Tat for TAR binding. As expected, HT1 also bound to endogenous 7SK snRNA.

These results confirm that HT1 competes with Tat for P-TEFb binding and keeps its kinase subunit CDK9 inactive, which reduces the amount of P-TEFb that is available for Tat to bring to TAR. Moreover, HT1 also binds to TAR and prevents Tat from binding it, consistent with the observation of a more potent inhibition by HT1 than by HT3. Two mechanisms are thus combined that prevent Tat from bringing active P-TEFb to TAR for successful HIV transcription elongation.

Expression of HT1 does not Impair Host Cell Gene Expression and Growth.

Since P-TEFb is a major transcription factor and regulates the expression of many cellular genes, we next assessed whether HT1 could impair host cell gene expression through inactivation of the kinase activity of CDK9. We first investigated how HT1 impacted the mRNA and protein expression levels of HEXIM1, a bona fide target of P-TEFb. Increasing amounts of m:HT1 were transiently expressed in 293T cells and did not change the expression level of HEXIM1 protein and mRNA.

The specificity of HT1 was further investigated by mRNA-seq analysis in 293T cells. Only 48 genes were differentially expressed upon ectopic expression of HT1, while knocking out CycT1 as a control impacted 1673 genes. A third of the genes impacted by HT1 expression corresponded to up-regulated non-coding RNAs, including 7SK (fold-change=1.1, padj=9.6E−06), an effect that may be due to a stabilization of these RNAs.

Finally, the impact of HT1 on cell growth was assessed using three T cell lines (CEM, MOLT4, and MT4) infected by a lentivirus expressing a triple Flag-epitope tagged HT1 (3f:HT1). Polyclonal population of HT1-expressing cells (C-HT, MO-HT, and MT-HT, respectively) was selected by puromycin and confirmed by BFP expression detected by FACS analysis and WB. Total viable cell count over time showed no difference in cellular growth rate between HT1-expressing and control cells. This confirmed that HT1 was specific to HIV inhibition, and that the few off-target effects had little impact on the metabolism of the cells.

Stable Expression of HT1 Inhibits HIV Reactivation and Replication.

To test the effect of HT1 on HIV-1 in T-lymphocyte derived cells, 3f:HT1 was stably expressed in HIV latent infection models 2D10 and J-Lat 9.2 cells (D-HT and L-HT cells respectively), which carry replication-defective, GFP-flagged HIV proviruses. In both 2D10 and J-Lat 9.2, the basal HIV transcription level is undetectable, as measured through GFP expression. Compounds such as the PKC agonist PMA, the histone deacetylase inhibitor SAHA or the BET bromodomain inhibitor JQ1 reactivate HIV transcription and increase GFP-positive cells detected by FACS analysis. 2D10 cells are more sensitive to HIV reactivation than J-Lat 9.2 cells, though they harbor a mutation in the N-terminal sequence of Tat (H13L). 2D10 and D-HT cells were incubated for 24 hrs with PMA (10 nM), SAHA (5 μM) or JQ1 (1 μM) and GFP positive cells were detected by FACS analysis. PMA, SAHA and JQ1 reactivated HIV from 65%, 71% and 58% less D-HT cells than from control 2D10 cells, respectively. Due to the limited HIV reactivation by SAHA and JQ1 in J-Lat 9.2 cells, we only treated them with PMA (100 nM), which induced a 12% increase in GFP expression. Stable expression of HT1 resulted in a 94% decrease of GFP expression in L-HT compared to J-Lat 9.2. Taken together, these results show that stable expression of HT1 significantly impairs HIV reactivation in latently infected T cells, consistent with previous observation that HT1 can inhibit HIV gene expression.

To investigate whether HT1 also inhibits HIV in a spreading infection, we infected the C-HT, MO-HT, and MT-HT cells with wild type HIV-1 NL43 virus, and collected the supernatants on 0, 2 and 4 days post infection (dpi) to assess viral production by Gag p24 ELISA. On 2 dpi, virus production was detected from CEM, MOLT4 and MT4 cells and reached around 20 (CEM, MT4) to 110 (MOLT4) ng/mL p24 concentration in the supernatant on 4 dpi. There was only 1.3 ng/mL, 5.1 and 12 ng/mL p24 concentration in the MT-HT, C-HT and MO-HT supernatants on 4 dpi, indicating that stable expression of HT1 reduced HIV replication by 75 to 95% in these cells. Single-round infection assays using HIV Env-pseudotyped replication-defective HIV, followed by measurement of proviral DNA by qPCR, indicated that early steps of HIV infection were not affected by HT1 expression. Together with the HIV reactivation assays, these results confirm that HT1 is a potent inhibitor of HIV gene expression and replication.

Discussion

In this study, we developed a new approach to block HIV transcription. We designed a chimera (HT1) containing the RNA-binding (ARM) and CDK9-inhibitory (ID) domains from the transcription regulator HEXIM1, and the P-TEFb-binding domain from the viral transactivator Tat (AD). Consistent with the respective properties of these domains in the context of their original proteins, HT1 competed with Tat for P-TEFb- and TAR-binding, and kept P-TEFb inactive. As a consequence, HT1 prevented Tat from bringing active P-TEFb to TAR for successful transcription elongation, as confirmed by the potent inhibition of HIV gene expression and replication. The use of a Tat-derived domain also conferred HT1 a high level of specificity, with little impact on host gene transcription and metabolism.

Herein is validated a new strategic approach in HIV therapy: we used the fundamental knowledge in the structure and function of proteins involved in Tat-dependent HIV transcription for a logical design of an inhibitory peptide. ID of HEXIM1 contains the PYNT motif which is critical for inhibition of CDK9 kinase activity. However, the C-terminal CycT1-binding domain (TBD) also contributes to the CDK9-inhibition by HEXIM1. Since the AD of Tat has a higher affinity to CycT1 than HEXIM1, replacing HEXIM1's TBD with Tat AD was expected to make HT1 able to compete with HEXIM1 for P-TEFb binding. Adding HEXIM1 ARM to HT1 also made it able to compete with Tat ARM for TAR binding. The three domains were thus needed for HT1 to efficiently compete with HEXIM1 and Tat, so that it potently inhibits P-TEFb-induced HIV transcription elongation. In addition, spatial arrangement of these domains was critical for the inhibitory activity, suggesting requirement for a precise placement of ID between the RNA- and P-TEFb-binding domains, as is naturally the case in HEXIM1. However, and as opposed as HEXIM1, HT1 did not need the C-terminal coiled-coil domain that is required for HEXIM1 dimerization and inhibitory activity. Indeed, we showed that HT1 successfully competed with Tat in both Tat/TAR/P-TEFb and HEXIM1/7SK/P-TEFb complexes, and kept bound P-TEFb inactive. HT1 was especially efficient in competing for TAR binding, which may be due to higher affinity for RNA through a longer and more basic ARM than Tat's. Since an efficient Tat/TAR/P-TEFb interaction involves the Tat-TAR recognition motif of CycT1, HT1 competing for P-TEFb binding may also contribute to the competition for TAR-binding. Our study hence demonstrated that HT1 properties precisely match assumptions derived from each piece of biochemical data on Tat/TAR/P-TEFb and HEXIM1/7SK/P-TEFb interactions. Precise design can therefore make peptide therapy more specific, and thus better tolerated than the small molecules most often used in cART, as confirmed by the only minor off-target effects of HT1 with no impact on cell growth. Reactivation assays from two distinct HIV latency models showed that HT1 efficiently inhibited HIV reactivation by a broad range of latency reversal agents. This efficiency was further confirmed in spreading HIV infection assays, where HT1 not only competed with Tat, but prevented Tat to be expressed from the first round of transcription of newly integrated proviruses. This positive feedback loop of inhibition resulted in efficient inhibition of HIV replication after multiple rounds of infection.

Multiple therapeutic applications are possible for HT1, from prevention to therapy and cure. HT1 domains were derived from the human protein HEXIM1 and from HIV-1 Tat, neither of which are immunogenic, suggesting that clinical use of the chimera would be well tolerated. A key focus should be on investigating feasible cell delivery and route of administration. In an era of multiple and potent cART options, acceptability of a potentially injection-based treatment would mostly depend on the frequency and duration of administration, and may especially be fit for HIV cure application as opposed to long-term use as cART. Diverse options are now at hand in the fast evolving field of peptide therapeutics, including injection or alternative delivery routes such as oral or transdermal. Gene therapy should also be considered, since we showed a positive inhibitory feedback loop in cells that stably expressed HT1 prior to HIV infection. In this model, HIV was virtually put into direct latency in newly infected cells, which in combination with blocking reactivation from pre-existing latently infected cells, could contribute to achieving a functional cure.

Finally, the design of such chimeras can be finely tuned to block other transcription factors that depend on P-TEFb. The CDK9-inhibiting module from HEXIM1 can be combined with functional domains from transactivator targets other than Tat, allowing this strategy to be applied to other pathologies, including inflammation or cancer. This study thus paves the way to multiple applications of transcription-targeted inhibition peptide therapy.

### Example 2. Chimeric Fusion Protein Utilizing TAT ARM

The THT fusion protein (SEQ ID NO: 7) comprises the TAT ARM (SEQ ID NO: 2), HEXIM CDK9 inhibitory domain (SEQ ID NO: 3), and TAT P-TEFb-binding domain (SEQ ID NO: 4). In luciferase assays, performed as in Example 1, THT potently inhibited HIV transcription.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A fusion protein,
wherein the fusion protein is configured for use in a method of inhibiting transcription mediated by P-TEFb and a selected target transcription factor;
wherein the fusion protein comprises:
a first element comprising a transcription factor recruitment element which facilitates interaction between the fusion protein and the selected target transcription factor;
a first linker sequence;
a second element comprising a CDK9 inhibiting domain that maintains the CDK9 subunit of P-TEFb in an inactive state;
a second linker sequence; and
a third element comprising a P-TEFb-binding domain that competes with the target transcription factor for P-TEFb binding.

2. The fusion protein of claim 1, wherein
the target transcription factor is selected from the group consisting of NFkB, cMyc, MyoD, a STAT-family transcription factor, Class II transactivator, Autoimmune Regulator, Eleven Nineteen Leukemia (ENL) or AF4 protein implicated in acute leukemia, an estrogen receptor, an androgen receptor, MEF2, and HTLV1 Tax.

3. The fusion protein of claim 1, wherein
the target transcription factor is HIV Tat.

4. The fusion protein of claim 3, wherein
the first element comprises an inhibitor of TAR/Tat complex formation or activity.

5. The fusion protein of claim 3, wherein
the first element comprises an arginine rich motif.

6. The fusion protein of claim 5, wherein
the first element comprises the HEXIM1 arginine rich motif.

7. The fusion protein of claim 5, wherein
the first element comprises a sequence having at least 95% sequence identity to SEQ ID NO: 1.

8. The fusion protein of claim 5, wherein
the first element comprises the HIV-1 TAT arginine rich motif.

9. The fusion protein of claim 3, wherein
the first element comprises a sequence having at least 95% sequence identity to SEQ ID NO: 2.

10. The fusion protein of claim 1, wherein
the second element comprises the CDK9 inhibiting element of HEXIM1.

11. The fusion protein of claim 1, wherein
the second element comprises a sequence having at least 95% sequence identity to SEQ ID NO: 3.

12. The fusion protein of claim 1, wherein
the third element comprises the P-TEFb-binding domain of HIV-1 TAT.

13. The fusion protein of claim 1, wherein
the third element comprises a sequence having at least 95% sequence identity to SEQ ID NO: 4.

14. The fusion protein of claim 1, wherein
the first linker sequence and/or the linker sequence is omitted.

15. The fusion protein of claim 1, wherein
the first linker sequence and/or the second linker sequence comprises a serine and glycine rich amino acid sequence.

16. The fusion protein of claim 1, wherein
the first linker sequence and/or the second linker sequence comprises one to twenty repeats of SEQ ID NO: 5.

17. The fusion protein of claim 3, wherein
the fusion protein comprises a sequence having at least 95% sequence identity to SEQ ID NO: 6.

18. The fusion protein of claim 3, wherein
the fusion protein comprises a sequence having at least 95% sequence identity to SEQ ID NO: 7.

19. The fusion protein of claim 1, wherein
the first element and the first linker are omitted.

20. The fusion protein of claim 19, wherein
the fusion protein comprises a sequence having at least 95% sequence identity to SEQ ID NO: 8.

21. The fusion protein of claim 1, wherein
the second element and the second linker are omitted.

22. The fusion protein of claim 21, wherein
the fusion protein comprises a sequence having at least 95% sequence identity to SEQ ID NO: 9.

23. A method of treating a condition in a subject in need of treatment therefor,
wherein P-TEFb interaction with a selected transcription factor is implicated in the condition, comprising
administering to the subject a pharmaceutically effective amount of a fusion protein, or a nucleic acid sequence coding therefor, wherein the fusion protein comprises:
a first element comprising a transcription factor recruitment element which facilitates interaction between the fusion protein and the selected transcription factor;
an optional first linker sequence;
a second element comprising a CDK9 inhibiting domain that maintains the CDK9 subunit of P-TEFb in an inactive state;
an optional second linker sequence; and
a third element comprising a P-TEFb-binding domain that competes with the target transcription factor for P-TEFb binding.

24. The method of claim 23, wherein,
the condition is selected from viral infection, cancer, cardiac hypertrophy, and an inflammatory condition.

25. The method of claim 24, wherein
the condition is HIV infection and the selected transcription factor is HIV Tat.

26. The method of claim 23, wherein
the first element and the first linker are omitted or the second element and the second linker are omitted.


SEQUENCE LISTING (12 sequences)

SEQ ID NO: 1 - protein, 28 aa, Homo sapiens
KKKHRRRPSKKKRHWKPYYKLTWEEKKK

SEQ ID NO: 2 - protein, 9 aa, Human immunodeficiency virus 1
RKKRRQRRR

SEQ ID NO: 3 - protein, 33 aa, Homo sapiens
RIRAEMFAKGQPVAPYNTTQFLMDDHDQEEPDL

SEQ ID NO: 4 - protein, 48 aa, Human immunodeficiency virus 1
MEPVDPYLEPWKHPGSQPRTACNNCYCKKCCFHCYACFTRKGLGISYG

SEQ ID NO: 5 - protein, 5 aa, Artificial Sequence
  Description of Artificial Sequence Synthetic peptide
GGGGS

SEQ ID NO: 6 - protein, 121 aa, Artificial Sequence
  Description of Artificial Sequence Synthetic polypeptide
KKKHRRRPSKKKRHWKPYYKLTWEEKKKFDEKQSLRASRIRAEMFAKGQPVAPYNTTQFLMDDHDQEEPDLVDMEPVDPYLEPWKHPGSQPRTACNNCYCKKCCFHCYACFTRKGLGISYG

SEQ ID NO: 7 - protein, 111 aa, Artificial Sequence
  Description of Artificial Sequence Synthetic polypeptide
MEQKLISEEDLRKKRRQRRRAPPGGGGSRIRAEMFAKGQPVAPYNTTQFLMDDHDQEEPDLVDMEPVDPYLEPWKHPGSQPRTACNNCYCKKCCFHCYACFTRKGLGISYG

SEQ ID NO: 8 - protein, 93 aa, Artificial Sequence
  Description of Artificial Sequence Synthetic polypeptide
FDEKQSLRASRIRAEMFAKGQPVAPYNTTQFLMDDHDQEEPDLVDMEPVDPYLEPWKHPGSQPRTACNNCYCKKCCFHCYACFTRKGLGISYG

SEQ ID NO: 9 - protein, 78 aa, Artificial Sequence
  Description of Artificial Sequence Synthetic polypeptide
KKKHRRRPSKKKRHWKPYYKLTWEEKKKVDMEPVDPYLEPWKHPGSQPRTACNNCYCKKCCFHCYACFTRKGLGISYG

SEQ ID NO: 10 - DNA, 372 bp, Artificial Sequence
  Description of Artificial Sequence Synthetic polynucleotide
atggaattcaagaaaaaacataggagacgcccgtccaagaagaagcggcattggaaaccgtactacaagctgacctgggaagagaagaaaaagttcgacgagaaacagagccttcgagcttcaaggatccgagccgagatgttcgccaagggccagccggtcgcgccctataacaccacgcagttcctcatggatgatcacgaccaggaggagccggatctcgtcgacatggagccagtagatccatatctagagccctggaagcatccaggaagtcagcctaggactgcttgtaacaattgctattgtaaaaagtgttgctttcattgctacgcgtgtttcacaagaaaaggcttaggcatctcctatggc

SEQ ID NO: 11 - DNA, 279 bp, Artificial Sequence
  Description of Artificial Sequence Synthetic polynucleotide
ttcgacgagaaacagagccttcgagcttcaaggatccgagccgagatgttcgccaagggccagccggtcgcgccctataacaccacgcagttcctcatggatgatcacgaccaggaggagccggatctcgtcgacatggagccagtagatccatatctagagccctggaagcatccaggaagtcagcctaggactgcttgtaacaattgctattgtaaaaagtgttgctttcattgctacgcgtgtttcacaagaaaaggcttaggcatctcctatggc

SEQ ID NO: 12 - DNA, 303 bp, Artificial Sequence
  Description of Artificial Sequence Synthetic polynucleotide
aggaagaagcggagacagcgacgaagagctcctcctggtggaggaggctctaggatccgagccgagatgttcgccaagggccagccggtcgcgccctataacaccacgcagttcctcatggatgatcacgaccaggaggagccggatctcgtcgacatggagccagtagatccatatctagagccctggaagcatccaggaagtcagcctaggactgcttgtaacaattgctattgtaaaaagtgttgctttcattgctacgcgtgtttcacaagaaaaggcttaggcatctcctatggctag